(19) United States
(12) Patent Application Publication
(10) Pub. No.: US 2021/0137820 A1
(43) Pub. Date: May 13, 2021

(54) COMPOSITION FOR CARING FOR AND/OR MAKING UP KERATIN MATERIALS

(71) Applicant: L'OREAL, Paris (FR)

(72) Inventors: Xia JIANG, Shanghai (CN); Yan WANG, Shanghai (CN)

(73) Assignee: L'OREAL, Paris (FR)

(21) Appl. No.: 16/957,532

(22) PCT Filed: Dec. 28, 2017
(86) PCT No.: PCT/CN2017/119403

§ 371 Date: Jun. 24, 2020

(51) Int. Cl. A61K 8/893; A61K 8/81; A61Q 19/00; A61Q 1/02

(52) U.S. Cl. CPC A61K 8/893; A61K 8/8152; A61K 2800/544; A61Q 1/02; A61K 2800/43; A61Q 19/00

(57) ABSTRACT

The present invention relates to a composition for caring for and/or making up keratin materials, comprising, in a continuous aqueous phase, (i) at least one associative copolymer of (meth)acrylic acid, and of (C₁-C₆)alkyl ester of (meth)acrylic acid; (ii) at least one film forming polymer chosen from vinyl polymer grafted with a carbosiloxane dentrimer; and (iii) at least one pigment.

### TECHNICAL FIELD

The present invention relates to the field of cosmetics, and especially to the field of compositions for caring for and/or making up keratin materials. Further, the present invention relates to the corresponding non-therapeutic cosmetic process involving use of same compositions.

The expression “compositions for caring for and/or making up keratin materials” according to the present invention is understood to mean cosmetic compositions different from rinse-off cleansing compositions.

In particular, they will be compositions for caring for and/or making up the skin of the face and/or of the body, especially facial skin.

### BACKGROUND ART

For centuries, consumers around the world are in search of novel cosmetic products for improving the appearance of keratin materials and especially the skin, in particular the optical cosmetic effect of the skin, such as brightening effect, coverage effect and radiance effect of the skin. Cosmetic compositions able to bring skin the optical effects as mentioned above are known, for example as foundation products containing colorants and oils in emulsion type, and particularly in water-in-oil emulsion type. Moreover, this cosmetic effect is also expected to be long lasting, due to the use routine of this type of products (using for all day long). However these foundation products are still not satisfying. For example, they may leave the skin dry after application. This may be due to their low capability to wrap large amounts of water.

Beside the cosmetic effect as mentioned above, consumers have increasing requirements on skin sensory properties. Among which, hydration is one of the most demanding requirements nowadays.

It is knowns that cosmetic products that contain high amount of aqueous phase can deliver a hydration sensory to the skin. Efforts have been made to formulate foundation products with high amount of aqueous phase, or in particular oil-in-water emulsions, oil dispersions type, in order to fulfill the need. However, the getting of watery texture is usually in contradiction with the desired cosmetic properties as described above, and furthermore in contradiction with the stability of cosmetic products of foundation type.

Therefore, formulating a composition for caring for and/or making up keratin materials with an expected cosmetic effect as well as a hydration sensory is still a difficulty the man skilled in the art is facing.

Besides, there is still a need for formulating a composition with the properties described above, and is stable over time.

### AIMS OF THE PRESENT INVENTION

The present invention aims at solving one or more of the problems mentioned above.

Therefore, in one aspect, it is necessary to formulate compositions, in particular makeup foundations, which are capable, on application, of providing a long lasting cosmetic effect on keratin materials, in particular the skin.

In another aspect, it is necessary to formulate compositions, in particular makeup foundations, which provide a good hydration effect to keratin materials, in particular the skin.

In another aspect, it is necessary to formulate compositions, in particular makeup foundations, which are stable over time.

### DISCLOSURE OF INVENTION

The inventors have discovered that the specific combination of (i) at least one specific associative copolymer of (meth)acrylic acid, and of (C₁-C₆)alkyl ester of (meth)acrylic acid, (ii) at least one silicone film former, and (iii) pigments can satisfy one or more of the abovementioned requirements.

The other subject of the present invention is a process for making up/caring for keratin materials, for example the skin, by applying to the keratin materials the composition of the present invention.

Throughout the description, including the claims, the term “comprising a” should be understood as being synonymous with “comprising at least one”, unless otherwise mentioned. Moreover, the expression “at least one” used in the present description is equivalent to the expression “one or more”.

In that which follows and unless otherwise indicated, the limits of a range of values are included within this range, in particular in the expressions “of between” and “ranging from . . . to . . . ”. Further, the terms “between . . . and . . . ” and “ranging from . . . to . . . ” should be understood as being inclusive of the limits, unless otherwise specified.

“Stable over time” is understood to mean compositions of the present invention which, after storage at all temperatures between 4° C. and 45° C. for 2 months, do not exhibit any macroscopic change in colour, smell or viscosity, any variation in pH or any variation in microscopic appearance.

By “keratin materials” we intend to mean human keratin materials such as skin, lips, and nails, in particular skin and lips, more preferably skin, particularly facial skin.

### DETAILED DESCRIPTION OF THE PRESENT INVENTION

The present invention relates to a composition for caring for and/or making up keratin materials, comprising, in a continuous aqueous phase,
(i) at least one associative copolymer of (meth)acrylic acid, and of (C₁-C₆)alkyl ester of (meth)acrylic acid;
(ii) at least one film forming polymer chosen from vinyl polymer grafted with a carbosiloxane dentrimer; and
(iii) at least one pigment.

### Associative Copolymer(s)

The composition according to the present invention includes (i) at least one associative copolymer of (meth)acrylic acid, and of (C₁-C₆)alkyl ester thereof.

Optionally two or more the (i) associative copolymer may be used in combination. Thus, a single type of the (i) associative copolymer or a combination of different types of the (i) associative copolymer may be used.

For the purpose of the present invention, the term “associative polymer” or “associative copolymer” refers to polymers or copolymers that are capable, in an aqueous medium, of reversibly combining with each other or with other molecules.

The associative copolymer more particularly comprises at least one hydrophilic part and at least one hydrophobic part.

For the purpose of the present invention, the term “hydrophobic group” means a radical or polymer with a saturated or unsaturated, linear or branched hydrocarbon-based chain, comprising at least 10 carbon atoms, preferably from 10 to 30 carbon atoms, in particular from 12 to 30 carbon atoms and more preferentially from 16 to 30 carbon atoms.

The associative copolymers disclosed herein can be partially or totally crosslinked with at least one standard crosslinking agent.

The at least one crosslinking agent can be chosen, for example, from polyunsaturated compounds, such as polyethylenically unsaturated compounds. For example, these compounds can be chosen from polyalkenyl ethers of sucrose, polyalkenyl ethers of polyols, diallyl phthalates, divinylbenzene, allyl (meth)acrylate, ethylene glycol di(meth)acrylate, methylenebisacrylamide trimethylolpropane tri(meth)acrylate, diallyl itaconate, diallyl fumarate, diallyl maleate, zinc (meth)acrylate, castor oil derivatives and polyol derivatives manufactured from unsaturated carboxylic acids.

For the purpose of the present invention, the associative copolymer of (meth)acrylic acid means that the copolymer comprises at least one acrylic acid unit or one methacrylic acid unit or a mixture thereof. The associative copolymer may comprise further units such as units formed by an alkyl ester of acrylic acid or methacrylic acid, preferably of acrylic acid, comprising less than 6 carbon atoms: for example, a C₁-C₄ alkylacrylate, for example, chosen from methyl acrylate, ethyl acrylate and butyl acrylate, called hereinafter “simple ester”.

According to a particular embodiment, the associative copolymer according to the present invention comprises at least one acrylic acid unit.

According to a further embodiment, the associative copolymer according to the present invention comprises at least one methacrylic acid unit.

According to another embodiment, the associative copolymer according to the present invention comprises at least an acrylic acid unit and a methacrylic acid unit.

In a particular embodiment, the associative copolymer may comprise another monomer that is different from (meth)acrylic acid and from (C₁-C₆)alkyl ester thereof.

For example, such monomer may be esters of ethylenically unsaturated hydrophilic monomers, such as (meth)acrylic acid or itaconic acid, and of polyoxyalkylenated fatty alcohols.

Preferably, the fatty alcohol moiety of the ester monomer can be linear or branched, preferably linear, saturated or unsaturated, preferably saturated, (C₁₂-C₃₀)fatty alcohol, and in particular (C₁₆-C₂₆) fatty alcohol. The polyoxyalkylene chain of the ester monomer preferentially consists of ethylene oxide units and/or propylene oxide units and even more particularly consists of ethylene oxide units. The number of oxyalkylene units generally ranges from 3 to 100, preferably from 7 to 50 and more preferably from 12 to 30.

According to an embodiment, the associative polymer (i) of the present invention preferably comes from the copolymerization between 1) and 2):

1) at least one ethylenically unsaturated mono or dicarboxylic acid monomer substituted by at least one, linear or branched, (C₁-C₁₀)alkyl group; and

2) at least one associative monomer which is an ester of formula (I):

A-O-(Alk-0)_{z}—(CH₂)_{w}—Rₐ (I)

formula (I) wherein:

A represents a ethylenically unsaturated acyclic residue, optionally containing an additional carboxylic group or it salt, wherein said additional carboxylic group may be esterified with a linear or branched (C₁-C₂₀)alkyl group;

Ra represents an alkyl a linear or branched (C₁-C₃₀)alkyl group, alkylaryl or arylalkyl group having from 1 to 30 carbon atoms wherein the alkyl group is linear or branched, preferably Ra represents (C₁-C₂₀)alkyl group, alkylphenyl or phenylakyl group having from 1 to 20 carbon atoms wherein the alkyl group is linear or branched;

Alk represents a linear or branched (C₁-C₆)alkylene group, particularly Alk represents —CH₂—CH(R_{b})— wherein R_{b} represents a hydrogen atom, or a (C₁-C₄)alkyl group such as methyl or ethyl group;

z is an integer comprised inclusively between 0 and 50;

w is an integer comprised inclusively between 0 and 30;

with the proviso that (I) contains at least on carboxyl group C(O)OH, or C(O)O⁻Q⁺ wherein Q⁺ represents cation chosen from an alkali metal, an alkaline earth metal, or ammonium;

By polymerization of 1) and 2), it must be understood a copolymerisation between at least one monomer 1) with at least one monomer 2).

According to an embodiment of the invention the copolymer comes from the polymerization between at least one ethylenically unsaturated mono or dicarboxylic acid monomer (1a) and at least one associative monomer which is an ester of formula (I) as defined herein before or (2a):

Formulas (1a) and (2a) wherein:

R′ₐ and R′_{b}, identical or different, represent a hydrogen atom, or a linear or branched (C₁-C₆)alkyl group, preferably R′ₐ and R′_{b} represent hydrogen atom;

R′_{c} and R″_{c}, identical or different, represent a hydrogen atom, or a linear or branched (C₁-C₆)alkyl group, a C(O)OX group, or a -alk-C(O)OX group wherein X represents a hydrogen atom, an alkali metal, alkaline earth metal, or ammonium and -alk- represents a (C₁-C₆)alkylene group such as methylene group, preferably R′_{c} and/or R″_{c} represent a hydrogen atom or a methyl group;

R₁ represents a hydrogen atom, an alkali metal, alkaline earth metal, or a (C₁-C₆)alkyl group;

R₂ represents a, linear or branched, (C₆-C₄₀)alkyl group, preferably a (C₁₀-C₃₀)alkyl group;

n is an integer comprised inclusively between 5 and 100, particularly between 10 and 50, more particularly between 20 and 40, preferably between 20 and 30 such as 25;

with the proviso that (1a) or (2a) contain at least on carboxyl group C(O)OH, or C(O)O-Q⁺ wherein Q⁺ represents cation chosen from an alkali metal, alkaline earth metal or ammonium;

Particularly R′ₐ, R′_{b} represent a hydrogen atom and R′_{c}, and R″_{c} represent a hydrogen atom or a methyl group and R₁ represents a hydrogen atom, an alkali metal, alkaline earth metal.

According to another variant R′ₐ, R′_{b}, and R′_{c}, represent a hydrogen atom and

[chemical structure]

R″_{c} represents a group -alk-C(O)OX such as —CH2-C(O)OX wherein X is as defined herein before.

According to a particular embodiment of the invention, the polymer i) contains units (Ia) and/or (I′a):

[chemical structure]

wherein

R′ₐ, R′_{b}, R′_{c}, and R″_{c} are as defined herein before;

x represents an integer, preferably more than 100, more preferably between 100 and 10000;

y represents an integer, preferably more than 100, more preferably between 100 and 10000;

and x+y represents an integer, preferably >200, more preferably between 200 and 20000.

According to a preferred embodiment, the associative copolymer (i) of the present invention has a molecular weight of more than 100000, preferably between 200000 and 8000000.

According to a preferred embodiment, in formula (Ia) and (I′a), R₁ represents a hydrogen atom, an alkali metal, or an alkaline earth metal.

As example of copolymer (1a)/(2a) as defined herein before, usable in the invention, we may mention: acrylates/palmeth-25 acrylate copolymer, such as the products commercially available from 3V under the trade name Synthalen® W2000, acrylates/beheneth-25 methacrylate copolymer, such as the products commercially available from Lubrizol under the trade name Novethix® L-10, or Volarest™ FL-LQ-(RB) sold by the company Croda; acrylates/steareth-20 methacrylate copolymer, such as the products commercially available from Rohm and Haas (Dow Chemical) under the trade name Aculyn™ 22 polymer, acrylates/steareth-20 itaconate copolymer, such as the products commercially available from AkzoNobel under the trade name Structure 2001, acrylates/ceteth-20 itaconate copolymer, such as the products commercially available from AkzoNobel under the trade name Structure 3001, acrylates/ceteth-20 methacrylate copolymer, acrylate/beheneth-25 itaconate copolymer, acrylate/palmeth-25 methacrylates copolymer, acrylate/steareth-50 acrylate copolymer, acrylates/palmeth-25 itaconate copolymer, such as the products commercially available from Sigma-3V under the trade name Polygel W 40, and mixtures thereof.

Among the above said polymers, acrylates/beheneth-25 methacrylate copolymer, and in particular the products sold by the company Croda under the tradename Volarest™ FL-LQ-(RB) is specially preferred.

The associative copolymer (i) described above are preferably used according to the invention in an amount which ranges from 0.05 to 2.5% by weight, preferably from 0.1 to 2.2% by weight, more preferably from 0.3 to 2.0% by weight, relative to the total weight of the composition.

Film Forming Polymer(s)

According to an embodiment, the composition of the present invention comprises at least one film forming polymer chosen from vinyl polymer grafted with a carbosiloxane dentrimer.

A vinyl polymer suitable for the preparation of a composition according to the invention comprises at least one carbosiloxane-dendrimer-derived unit.

The vinyl polymer may have, in particular, a backbone and at least one side chain, which comprises a carbosiloxane-dendrimer-derived unit having a carbosiloxane dendrimer structure.

The term “carbosiloxane dendrimer structure” in the context of the present invention represents a molecular structure possessing branched groups having high molecular masses, said structure having a high regularity in the radial direction starting from the linkage to the backbone. Such carbosiloxane dendrimer structures are described in the form of a highly branched siloxane-silylalkylene copolymer in the Japanese patent application made available to public inspection Kokai 9-171 154.

A vinyl polymer according to the invention may contain carbosiloxane-dendrimer-derived units which can be represented by the following general formula:

[chemical structure]

in which R¹ represents an aryl group or an alkyl group having from 1 to 10 carbon atoms, and Xⁱ represents a silylalkyl group which, when i=1, is represented by the formula:

[chemical structure]

in which R¹ is as defined above, R² represents an alkylene group having from 2 to 10 carbon atoms, R³ represents an alkyl group having from 1 to 10 carbon atoms, Xⁱ⁺¹ represents a hydrogen atom, an alkyl group having from 1 to 10 carbon atoms, an aryl group, or the silylalkyl group as defined above with i=i+1; i is an integer from 1 to 10 which represents the generation of said silylalkyl group, and a¹ is an integer from 0 to 3; Y represents a radical-polymerizable organic group selected from:
organic groups containing a methacrylic group or an acrylic group and which are represented by the formulae:

[chemical structure]

in which R⁴ represents a hydrogen atom or an alkyl group, R⁵ represents an alkylene group having from 1 to 10 carbon atoms, such as a methylene group, an ethylene group, a propylene group or a butylene group, the methylene group and the propylene group being preferred; and
organic groups containing a styryl group and which are represented by the formula:

[chemical structure]

in which R⁶ represents a hydrogen atom or an alkyl group, R⁷ represents an alkyl group having from 1 to 10 carbon atoms, such as a methyl group, an ethyl group, a propyl group or a butyl group, the methyl group being preferred, R⁸ represents an alkylene group having from 1 to 10 carbon atoms, such as a methylene group, an ethylene group, a propylene group or a butylene group, the ethylene group being preferred, b is an integer from 0 to 4, and c is 0 or 1 such that, if c is 0, —(R⁸)_{c}— represents a bond.

According to one embodiment, R¹ may represent an aryl group or an alkyl group having from 1 to 10 carbon atoms. The alkyl group may preferably be represented by a methyl group, an ethyl group, a propyl group, a butyl group, a pentyl group, an isopropyl group, an isobutyl group, a cyclopentyl group or a cyclohexyl group. The aryl group may preferably be represented by a phenyl group and a naphthyl group. Methyl and phenyl groups are more particularly preferred, and the methyl group is preferred above all.

A vinyl polymer having at least one carbosiloxane-dendrimer-derived unit has a molecular side chain containing a carbosiloxane dendrimer structure, and can be derived from the polymerization:
(A) of 0 to 99.9 parts by weight of a vinyl monomer; and
(B) of 100 to 0.1 parts by weight of a carbosiloxane dendrimer containing a radical-polymerizable organic group, represented by the general formula:

[chemical structure]

in which Y represents a radical-polymerizable organic group, R1 represents an aryl group or an alkyl group having from 1 to 10 carbon atoms, and Xi represents a silylalkyl group which, when i=1, is represented by the formula:

[chemical structure]

in which R¹ is as defined above, R² represents an alkylene group having from 2 to 10 carbon atoms, R³ represents an alkyl group having from 1 to 10 carbon atoms, Xⁱ⁺¹ represents a hydrogen atom, an alkyl group having from 1 to 10 carbon atoms, an aryl group, or the silylalkyl group as defined above with i=i+1; i is an integer from 1 to 10 which represents the generation of said silylalkyl, and aⁱ is an integer from 0 to 3;

where said radical-polymerizable organic group contained in the component (B) is selected from:

organic groups containing a methacrylic group or an acrylic group and which are represented by the formulae:

[chemical structure]

in which R⁴ represents a hydrogen atom or an alkyl group, R⁵ represents an alkylene group having from 1 to 10 carbon atoms; and
organic groups containing a styryl group and which are represented by the formula:

[chemical structure]

in which R⁶ represents a hydrogen atom or an alkyl group, R⁷ represents an alkyl group having from 1 to 10 carbon atoms, R⁸ represents an alkylene group having from 1 to 10 carbon atoms, b is an integer from 0 to 4, and c is 0 or 1, such that, if c is 0, —(R⁸)_{c}— represents a bond.

The monomer of vinyl type that is the component (A) in the vinyl polymer is a monomer of vinyl type which contains a radical-polymerizable vinyl group.

There is no particular limitation as regards such a monomer.

The following are examples of this vinyl-type monomer: methyl methacrylate, ethyl methacrylate, n-propyl methacrylate, isopropyl methacrylate, or a methacrylate of a lower alkyl analog; glycidyl methacrylate; butyl methacrylate, butyl acrylate, n-butyl methacrylate, isobutyl methacrylate, tert-butyl acrylate, tert-butyl methacrylate, n-hexyl methacrylate, cyclohexyl methacrylate, 2-ethylhexyl acrylate, 2-ethylhexyl methacrylate, octyl methacrylate, lauryl methacrylate, stearyl acrylate, stearyl methacrylate, or a higher-analog methacrylate; vinyl acetate, vinyl propionate, or a vinyl ester of a lower fatty acid analog; vinyl caproate, vinyl 2-ethylhexoate, vinyl laurate, vinyl stearate, or an ester of a higher fatty acid analog; styrene, vinyltoluene, benzyl methacrylate, phenoxyethyl methacrylate, vinylpyrrolidone, or similar vinyl aromatic monomers; methacrylamide, N-methylol¬methacrylamide, N-methoxy¬methylmethacrylamide, isobutoxy¬methoxy-methacrylamide, N,N-dimethyl¬methacrylamide, or similar monomers of vinyl type containing amide groups; hydroxyethyl methacrylate, hydroxypropyl methacrylate, or similar monomers of vinyl type containing hydroxyl groups; acrylic acid, methacrylic acid, itaconic acid, crotonic acid, fumaric acid, maleic acid, or similar monomers of vinyl type containing a carboxylic acid group; tetrahydrofurfuryl methacrylate, butoxyethyl methacrylate, ethoxydiethylene glycol methacrylate, polyethylene glycol methacrylate, polypropylene glycol monomethacrylate, hydroxybutyl vinyl ether, cetyl vinyl ether, 2-ethylhexyl vinyl ether, or a similar monomer of vinyl type with ether bonds; methacryloxypropyltrimethoxysilane, polydimethylsiloxane containing a methacrylic group on one of its molecular ends, polydimethylsiloxane containing a styryl group on one of its molecular ends, or a similar silicone compound containing unsaturated groups; butadiene; vinyl chloride; vinylidene chloride; methacrylonitrile; dibutyl fumarate; anhydrous maleic acid; anhydrous succinic acid; methacryl glycidyl ether; an organic salt of an amine, an ammonium salt, and an alkali metal salt of methacrylic acid, of itaconic acid, of crotonic acid, of maleic acid or of fumaric acid; a radical-polymerizable unsaturated monomer containing a sulfonic acid group such as a styrenesulfonic acid group; a quaternary ammonium salt derived from methacrylic acid, such as 2-hydroxy-3-methacryloxy-propyltrimethylammonium chloride; and a methacrylic acid ester of an alcohol containing a tertiary amine group, such as a methacrylic acid ester of diethylamine.

Multifunctional monomers of vinyl type may also be used.

The following represent examples of such compounds: trimethylolpropane trimethacrylate, pentaerythritol trimethacrylate, ethylene glycol dimethacrylate, tetraethylene glycol dimethacrylate, polyethylene glycol dimethacrylate, 1,4-butanediol dimethacrylate, 1,6-hexane¬diol dimethacrylate, neopentyl glycol dimethacrylate, trimethylol¬propanetrioxyethyl methacrylate, tris(2-hydroxyethyl)isocyanurate dimethacrylate, tris(2-hydroxyethyl)isocyanurate trimethacrylate, polydimethylsiloxane capped with styryl groups containing divinylbenzene groups on both ends, or similar silicone compounds containing unsaturated groups.

A carbosiloxane dendrimer, which is the component (B), can be represented by the following formula:

[chemical structure]

in which Y represents a radical-polymerizable organic group as defined above.

The following represent the preferred examples of a radical-polymerizable organic group Y: an acryloxymethyl group, a 3-acryloxypropyl group, a methacryloxymethyl group, a 3-methacryloxypropyl group, a 4-vinylphenyl group, a 3-vinylphenyl group, a 4-(2-propenyl)phenyl group, a 3-(2-propenyl)phenyl group, a 2-(4-vinylphenyl)ethyl group, a 2-(3-vinylphenyl)ethyl group, a vinyl group, an allyl group, a methallyl group and a 5-hexenyl group.

R¹ is as defined above.

Xⁱ represents a silylalkyl group which is represented by the following formula, when i is equal to one:

[chemical structure]

in which R¹ is as defined above.

R² represents an alkylene group having from 2 to 10 carbon atoms, such as an ethylene group, a propylene group, a butylene group, a hexylene group, or a similar linear alkylene group; a methylmethylene group, a methylethylene group, a 1-methylpentylene group, a 1,4-dimethylbutylene group, or a similar branched alkylene group.

Ethylene, methylethylene, hexylene, 1-methylpentylene and 1,4-dimethylbutylene groups are preferred above all.

R³ represents an alkyl group having from 1 to 10 carbon atoms, such as methyl, ethyl, propyl, butyl and isopropyl groups.

Xⁱ⁺¹ represents a hydrogen atom, an alkyl group having from 1 to 10 carbon atoms, an aryl group, or the silylalkyl group with i=i+1.

aⁱ is an integer from 0 to 3, an i is an integer from 1 to 10 which indicates the generation number, which represents the number of repetitions of the silylalkyl group.

For example, when the generation number is equal to one, the carbosiloxane dendrimer may be represented by the first general formula shown below, in which Y, R¹, R² and R³ are as defined above, R¹² represents a hydrogen atom or is identical to R¹; a¹ is identical to aⁱ. Preferably, the mean total number of OR³ groups in a molecule is within the range from 0 to 7.

When the generation number is equal to 2, the carbosiloxane dendrimer may be represented by the second general formula shown below, in which Y, R¹, R², R³ and R¹² are the same as defined above; a¹ and a² represent the aⁱ of the indicated generation. Preferably, the mean total number of OR³ groups in a molecule is in the range from 0 to 25.

When the generation number is equal to 3, the carbosiloxane dendrimer is represented by the third general formula shown below, in which Y, R¹, R², R³ and R¹² are the same as defined above; a¹, a² and a³ represent the aⁱ of the indicated generation. Preferably, the mean total number of OR³ groups in a molecule is in the range from 0 to 79.

[chemical structure]

A carbosiloxane dendrimer that contains a radical-polymerizable organic group may be represented by the following mean structural formulae:

[chemical structure]

The carbosiloxane dendrimer may be produced according to the process for producing a branched silalkylene siloxane described in Japanese patent application Hei 9-171154.

For example, it may be prepared by subjecting an organosilicon compound containing a hydrogen atom linked to a silicon atom, represented by the following general formula:

[chemical structure]

and an organosilicon compound containing an alkenyl group, to a hydrosilylation reaction.

In the above formula, the organosilicon compound may be represented by 3-methacryloxypropyltris(dimethylsiloxy)silane, 3-acryloxypropyltris- may be selected from polymers such that the carbosiloxane-dendrimer-derived unit is (dimethylsiloxy)silane, and 4-vinylphenyltris(dimethylsiloxy)silane. The organosilicon compound that contains an alkenyl group may be represented by vinyltris(trimethylsiloxy)silane, vinyltris(dimethylphenylsiloxy)silane, and 5-hexenyltris(trimethylsiloxy)silane.

The hydrosilylation reaction is carried out in the presence of a chloroplatinic acid, a complex of vinylsiloxane and of platinum, or a similar transition metal catalyst.

A vinyl polymer having at least one carbosiloxane-dendrimer-derived unit may be selected from polymers such that the unit of a derivative of a carbosiloxane dendrimer is a carbosiloxane dendrimer structure represented by formula (I):

[chemical structure]

in which Z is a divalent organic group, “p” is 0 or 1, R¹ is an aryl or alkyl group having from 1 to 10 carbon atoms and Xⁱ is a silylalkyl group represented by formula (II):

[chemical structure]

in which R¹ is as defined above, R² is an alkylene group having from 1 to 10 carbon atoms, R³ is an alkyl group having from 1 to 10 carbon atoms, and Xⁱ⁺¹ is a group selected from the group comprising hydrogen atoms, aryl groups and alkyl groups having up to 10 carbon atoms, and silylalkyl groups Xⁱ where the “i” is an integer from 1 to 10 indicating the generation of the silylalkyl group beginning in each carbosiloxane dendritic structure with a value of 1 for the Xⁱ group in formula (I) and the index “aⁱ” is an integer from 0 to 3.

In a vinyl polymer having at least one carbosiloxane-dendrimer-derived unit, the polymerization ratio between the components (A) and (B), in terms of the weight ratio between (A) and (B), may be within a range from 0/100 to 99.9/0.1, or even from 0.1/99.9 to 99.9/0.1, and preferably within a range from 1/99 to 99/1. A ratio between the components (A) and (B) of 0/100 means that the compound becomes a homopolymer of component (B).

A vinyl polymer having at least one carbosiloxane-dendrimer-derived unit may be obtained by copolymerization of the components (A) and (B), or by polymerization of the component (B) alone.

The polymerization may be a free-radical polymerization or an ionic polymerization, but free-radical polymerization is preferred.
The polymerization may be carried out by bringing about a reaction between the components (A) and (B) in a solution for a period of from 3 to 20 hours in the presence of a radical initiator at a temperature of from 50° C. to 150° C.

A suitable solvent for this purpose is hexane, octane, decane, cyclohexane or a similar aliphatic hydrocarbon; benzene, toluene, xylene or a similar aromatic hydrocarbon; diethyl ether, dibutyl ether, tetrahydrofuran, dioxane, or similar ethers; acetone, methyl ethyl ketone, methyl isobutyl ketone, diisobutyl ketone, or similar ketones; methyl acetate, ethyl acetate, butyl acetate, isobutyl acetate or similar esters; methanol, ethanol, isopropanol, butanol or similar alcohols; octamethylcyclotetrasiloxane, decamethylcyclopentasiloxane, hexamethyldisiloxane, octamethyltrisiloxane or a similar organosiloxane oligomer.

A radical initiator may be any compound known in the art for standard free-radical polymerization reactions. The specific examples of such radical initiators are 2,2′-azobis(isobutyronitrile), 2,2′-azobis(2-methylbutyronitrile), 2,2′-azobis(2,4-dimethylvaleronitrile) or similar compounds of azobis type; benzoyl peroxide, lauroyl peroxide, tert-butyl peroxybenzoate, tert-butyl peroxy-2-ethylhexanoate or a similar organic peroxide. These radical initiators may be used alone or in a combination of two or more. The radical initiators may be used in an amount of from 0.1 to 5 parts by weight per 100 parts by weight of the components (A) and (B). A chain-transfer agent may be added. The chain-transfer agent may be 2-mercaptoethanol, butyl mercaptan, n-dodecyl mercaptan, 3-mercaptopropyltrimethoxysilane, a polydimethylsiloxane containing a mercaptopropyl group, or a similar compound of mercapto type; methylene chloride, chloroform, carbon tetrachloride, butyl bromide, 3-chloropropyltrimethoxysilane or a similar halogenated compound.

In the production of the polymer of vinyl type, after polymerization, the residual unreacted vinyl monomer may be removed under conditions of heating under vacuum.

To facilitate the preparation of the mixture of the starting material of cosmetic products, the number-average molecular mass of the vinyl polymer containing a carbosiloxane dendrimer may be selected within the range of between 3000 and 2 000 000, preferably between 5000 and 800 000. It may be a liquid, a gum, a paste, a solid, a powder or any other form. The preferred forms are solutions constituted of the dilution, in solvents, of a dispersion or of a powder.

The vinyl polymer may be a dispersion of a polymer of vinyl type having a carbosiloxane dendrimer structure in its side molecular chain, in a liquid such as a silicone oil, an organic oil, an alcohol or water.

The silicone oil may be a dimethylpolysiloxane with the two molecular ends capped with trimethylsiloxy groups, a copolymer of methylphenylsiloxane and of dimethylsiloxane having the two molecular ends capped with trimethylsiloxy groups, a copolymer of methyl-3,3,3-trifluoropropylsiloxane and of dimethylsiloxane having the two molecular ends capped with trimethylsiloxy groups, or similar unreactive linear silicone oils, and also hexamethylcyclotrisiloxane, octamethylcyclotetrasiloxane, decamethylcyclopentasiloxane, dodecamethylcyclohexasiloxane, or a similar cyclic compound. In addition to the unreactive silicone oils, modified polysiloxanes containing functional groups such as silanol groups, amino groups and polyether groups on the ends or within the side molecular chains may be used. The organic oils may be isododecane, liquid paraffin, isoparaffin, hexyl laurate, isopropyl myristate, myristyl myristate, cetyl myristate, 2-octyldodecyl myristate; isopropyl palmitate, 2-ethylhexyl palmitate, butyl stearate, decyl oleate, 2-octyldodecyl oleate, myristyl lactate, cetyl lactate, lanolin acetate, stearyl alcohol, cetostearyl alcohol, oleyl alcohol, avocado oil, almond oil, olive oil, cocoa oil, jojoba oil, gum oil, sunflower oil, soybean oil, camelia oil, squalane, castor oil, cotton seed oil, coconut oil, egg yolk oil, propylene glycol monooleate, neopentyl glycol 2-ethylhexanoate, or a similar glycol ester oil; triglyceryl isostearate, the triglyceride of a fatty acid of coconut oil, or a similar oil of a polyhydric alcohol ester; polyoxyethylene lauryl ether, polyoxypropylene cetyl ether or a similar polyoxyalkylene ether.

The alcohol may be of any type that is suitable for use in combination with a cosmetic product starting material. For example, it may be methanol, ethanol, butanol, isopropanol or similar lower alcohols.

A solution or a dispersion of the alcohol should have a viscosity in the range from 10 to 10⁹ mPa at 25° C. To improve the sensory use properties in a cosmetic product, the viscosity should be within the range from 100 to 5×10⁸ mPa·s.

The solutions and the dispersions may be readily prepared by mixing a vinyl polymer having at least one carbosiloxane-dendrimer-derived unit with a silicone oil, and organic oil, an alcohol or water. The liquids may be present in the step of polymerization of a vinyl polymer having at least one carbosiloxane-dendrimer-derived unit. In this case, the unreacted residual vinyl monomer should be completely removed by heat treatment of the solution or dispersion under atmospheric pressure or reduced pressure.

In the case of a dispersion, the dispersity of the polymer of vinyl type may be improved by adding a surfactant.

Such a surfactant may be hexylbenzenesulfonic acid, octylbenzenesulfonic acid, decylbenzenesulfonic acid, dodecylbenzenesulfonic acid, cetylbenzenesulfonic acid, myristylbenzenesulfonic acid or anionic surfactants such as sodium salts of these acids; octyltrimethylammonium hydroxide, dodecyltrimethylammonium hydroxide, hexadecyltrimethylammonium hydroxide, octyldimethylbenzylammonium hydroxide, decyl-dimethylbenzylammonium hydroxide, dioctadecyldimethylammonium hydroxide, tallow-trimethylammonium hydroxide, coconut oil-trimethylammonium hydroxide, or a similar cationic surfactant; a polyoxyalkylene alkyl ether, a polyoxyalkylenealkylphenol, a polyoxyalkylene alkyl ester, the sorbitol ester of polyoxyalkylene, polyethylene glycol, polypropylene glycol, an ethylene oxide additive of diethylene glycol trimethylnonanol, and nonionic surfactants of polyester type, and also mixtures.

In addition, the solvents and the dispersions may be combined with iron oxide suitable for use with cosmetic products, or a similar pigment, and also zinc oxide, titanium oxide, silicon oxide, mica, talc or similar inorganic oxides in powder form. In the dispersion, a mean particle diameter of the polymer of vinyl type may be within a range of between 0.001 and 100 microns, preferably between 0.01 and 50 microns. This is because, outside the recommended range, a cosmetic product mixed with the emulsion will not have a nice enough feel on the skin or to the touch, or sufficient spreading properties or a pleasant feel.

A vinyl polymer contained in the dispersion or the solution may have a concentration in a range of between 0.1% and 95% by weight, preferably between 5% and 85% by weight. However, to facilitate the handling and the preparation of the mixture, the range should preferably be between 10% and 75% by weight.

A vinyl polymer suitable for the invention may also be one of the polymers described in the examples of application EP 0 963 751.

According to one preferred embodiment, a vinyl polymer grafted with a carbosiloxane dendrimer may be derived from the polymerization:

(A) of 0.1 to 99 part(s) by weight of one or more acrylate or methacrylate monomer(s); and

(B) of 100 to 0.1 part(s) by weight of an acrylate or methacrylate monomer of a tri[tri(trimethylsiloxy)silylethyldimethylsiloxy]silylpropyl carbosiloxane dendrimer.

According to one embodiment, a vinyl polymer having at least one carbosiloxane-dendrimer-derived unit may comprise a unit derived from a tri[tri(trimethylsiloxy)silylethyldimethylsiloxy]silylpropyl carbosiloxane dendrimer corresponding to one of the formulae:

[chemical structure]

According to one preferred embodiment, a vinyl polymer having at least one carbosiloxane-dendrimer-derived unit, used in the invention, comprises at least one butyl acrylate monomer.

According to one embodiment, a vinyl polymer may also comprise at least one organofluorine group.

Structures in which the polymerized vinyl units constitute the backbone and carbosiloxane dendritic structures and also organofluorine groups are attached to side chains are particularly preferred.

The organofluorine groups may be obtained by replacing with fluorine atoms all or some of the hydrogen atoms of methyl, ethyl, propyl, isopropyl, butyl, isobutyl, tert-butyl, pentyl, neopentyl, hexyl, cyclohexyl, heptyl, octyl, nonyl, decyl, undecyl, dodecyl, tridecyl, tetradecyl, hexadecyl and octadecyl groups and other alkyl groups having from 1 to 20 carbon atoms, and also alkyloxyalkylene groups having from 6 to 22 carbon atoms.

The groups represented by the formula: —(CH₂)ₓ—(CF₂)_{y}—R¹³ are suggested by way of examples of fluoroalkyl groups obtained by substituting fluorine atoms for hydrogen atoms of alkyl groups. In the formula, the index “x” is 0, 1, 2 or 3 and “y” is an integer from 1 to 20. R¹³ is an atom or a group selected from a hydrogen atom, a fluorine atom, —(CH(CF₃)₂— or CF(CF₃)₂. Such fluorine-substituted alkyl groups are exemplified by linear or branched polyfluoroalkyl or perfluoroalkyl groups represented by the formulae given below:

—CF₃, —C₂F₅, -nC3F7, —CF(CF₃)₂, -nC₄F₉, CF₂CF(CF₃)₂, -nC₅F₁₁, -nC₆F₁₃, -nC₈F₁₇, CH₂CF₃, —(CH(CF₃)₂, CH₂CH(CF₃)₂—CH₂(CF₂)₂F, —CH₂(CF₂)₃F, —CH₂(CF₂)₄F, CH₂(CF₂)₆F, CH₂(CF₂)₈F, —CH₂CH₂CF₃, —CH₂CH₂(CF₂)₂F, —CH₂CH₂(CF₂)₃F, —CH₂CH₂(CF₂)₄F, —CH₂CH₂(CF₂)₆F, —CH₂CH₂(CF₂)₈F, —CH₂CH₂(CF₂)₁₀F, —CH₂CH₂(CF₂)₁₂F, CH₂CH₂(CF₂)₁₄F, —CH₂CH₂(CF₂)₁₆F, —CH₂CH₂CH₂CF₃, —CH₂CH₂CH₂(CF₂)₂F, —CH₂CH₂CH₂(CF₂)₂H—CH₂(CF₂)₄H, and —CH₂CH₂(CF₂)₃H.

The groups represented by —CH₂CH₂—(CF₂)ₘ—CFR¹⁴—[OCF₂CF(CF₃)]ₙ—OC₃F₇ are suggested as fluoroalkyloxyfluoroalkylene groups obtained by substituting fluorine atoms for hydrogen atoms of alkyloxyalkylene groups. In the formula, the index “m” is 0 or 1, “n” is 0, 1, 2, 3, 4 or 5, and R^{H} is a fluorine atom or CF₃. Such fluoroalkyloxyfluoroalkylene groups are exemplified by the perfluoroalkyloxyfluoroalkylene groups represented by the formulae given below:

—CH₂CH₂CF(CF₃)—[OCF₂CF(CF₃)]ₙ—OC₃F₇,
—CH₂CH₂CF₂CF₂—[OCF₂CF(CF₃)]ₙ—OC₃F₇.

The number-average molecular weight of the vinyl polymer used in the present invention may be between 3000 and 2 000 000, and more preferably between 5000 and 800 000.

This type of fluorinated vinyl polymer may be obtained by addition:
of a vinyl monomer (B) not containing any organofluorine groups in the molecule,
to a vinyl monomer containing organofluorine groups in the molecule (A), and
a carbosiloxane dendrimer (C) containing radical-polymerizable organic groups represented by general formula (III):

[chemical structure]

in which Y is a radical-polymerizable organic group and R¹ and X¹ are as defined above, and by subjecting them to a copolymerization.

Thus, according to one embodiment, a composition of the invention may comprise a vinyl polymer having at least one carbosiloxane-dendrimer-derived unit and which is derived from the copolymerization:

(A) of vinyl monomers containing organofluorine groups in the molecule,

(B) optionally of vinyl monomers not containing any organofluorine groups in the molecule, and

(C) of carbosiloxane dendrimers having radical-polymerizable organic groups represented by general formula (III):

[chemical structure]

in which Y is a radical-polymerizable organic group, R¹ is an aryl or alkyl group having from 1 to 10 carbon atoms and Xⁱ is a silylalkyl group represented by formula (II) below:

[chemical structure]

in which R¹ is as above, R² is an alkylene group having from 1 to 10 carbon atoms, R³ is an alkyl group having from 1 to 10 carbon atoms, and Xⁱ⁺¹ is a group selected from the group comprising hydrogen atoms, aryl groups and alkyl groups having up to 10 carbon atoms, and silylalkyl groups Xⁱ mentioned above where the “i” is an integer from 1 to 10 indicating the generation of said silylalkyl group beginning in each carbosiloxane dendritic structure with a value of 1 for the group Xⁱ in formula (III), and the index “aⁱ” is an integer from 0 to 3, said vinyl polymer having a copolymerization ratio of the component (A) to the component (B) of 0.1 to 100:99.9 to 0% by weight, and a copolymerization ratio of the sum of the component (A) and of the component (B) to the component (C) of 0.1 to 99.9:99.9 to 0.1% by weight.

The vinyl monomers (A) containing organofluorine groups in the molecule are preferably monomers represented by the general formula: (CH²)═CR¹⁵COOR^{f}.

In the formula, R¹⁵ is a hydrogen atom or a methyl group, R^{f} is an organofluorine group exemplified by the fluoroalkyl and fluoroalkyloxyfluoroalkylene groups described above. The compounds represented by the formulae given below are suggested by way of specific examples of the component (A). In the formula given below, “z” is an integer from 1 to 4.

CH₂═CCH₃COO—CF₃, CH₂═CCH₃COO—C₂F₅, CH₂—CCH₃COO-nC₃F₇, CH₂═CCH₃COO—CF(CF₃)₂, CH₂CCH₃COO-nC₄F₉, CH₂═CCH₃COO—CF(CF₃)₂, CH₂═CCH₃COO-nC₅F₁, CH₂═CCH₃COO-nC₆F₁₃, CH₂═CCH₃COO-nC₈F₁₇, CH₂═CCH₃COO—CH₂CF₃, CH₂═CCH₃COO—CH(CF₃)₂, CH₂═CCH₃COO—CH₂CH(CF₃)₂, CH₂═CCH₃COO—CH₂ (CF₂)₂F, CH₂═CCH₃COO—CH₂(CF₂)₂F, CH₂═CCH₃COO—CH₂(CF₂)₄F, CH₂═CCH₃COO—CH₂(CF₂)₆F, CH₂═CCH₃COO—CH₂(CF₂)₈F, CH₂═CCH₃COO—CH₂CH₂CF₃, CH₂—CCH₃COO—CH₂CH₂(CF₂)₂F, CH₂═CCH₃COO—CH₂CH₂(CF₂)₃F, CH₂═CCH₃COO—CH₂CH₂(CF₂)₄F, CH₂═CCH₃COO—CH₂CH₂(CF₂)₆F, CH₂═CCH₃COO—CH₂CH₂(CF₂)₈F, CH₂═CCH₃COO—CH₂CH₂(CF₂)₁₀F, CH₂═CCH₃COO—CH₂CH₂(CF₂)₁₂F, CH₂═CCH₃COO—CH₂CH₂(CF₂)₁₄F, CH₂═CCH₃COO—CH₂—CH₂—(CF₂)₁₆F, CH₂═CCH₃COO—CH₂CH₂CH₂CF₃, CH₂═CCH₃COO—CH₂CH₂CH₂(CF₂)₂F, CH₂═CCH₃COO—CH₂CH₂CH₂ (CF₂)₂H, CH₂═CCH₃COO—CH₂ (CF₂)₄H, CH₂═CCH₃COO—(CF₂)₃H, CH₂═CCH₃COO—CH₂CH₂CF(CF₃)—[OCF₂—CF(CF₃)]z-OC₃F₇, CH₂═CCH₃COO—CH₂CH₂CF₂CF₂—[OCF₂—CF(CF₃)]z-OC₃F₇, CH₂═CHCOO—CF₃, CH2═CHCOO—C₂F₅, CH₂═CHCOO-nC₃F₇, CH₂═CHCOO—CF(CF₃)₂, CH₂═CHCOO-nC₄F₉, CH₂═CHCOO—CF₂CF(CF₃)₂, CH₂═CHCOO-nC₅F₁₁, CH₂═CHCOO-nC₆F₁₃, CH₂═CHCOO-nC₈F₁₇, CH₂═CHCOO—CH₂CF₃, CH₂═CHCOO—CH(CF₃)₂, CH₂═CHCOO—CH₂CH(CF₃)₂, CH₂═CHCOO—CH₂(CF₂)₂F, CH₂═CHCOO—CH₂(CF₂)₃F, CH₂═CHCOO—CH₂(CF₂)₄F, CH₂═CHCOO—CH₂(CF₂)₆F, CH₂═CHCOO—CH₂ (CF₂)₈F, CH₂═CHCOO—CH₂CH₂CF₃, CH₂═CHCOO—CH₂CH₂ (CF₂)₂F, CH₂═CHCOO—CH₂CH₂(CF₂)₃F, CH₂═CHCOO—CH₂CH₂(CF₂)₄F, CH₂═CHCOO—CH₂CH₂(CF₂)₆F, CH₂═CHCOO—CH₂CH₂(CF₂)₈F, CH₂═HCOO—CH₂CH₂(CF₂)₁₀F, CH₂—CHCOO—CH₂CH₂—(CF₂)₁₂F, CH₂═CHCOO—CH₂CH₂(CF₂)₁₄F, CH₂═CHCOO—CH₂CH₂(CF₂)₁₆F, CH₂═CHCOO—CH₂CH₂CH₂CF₃, CH₂═CHCOO—CH₂CH₂CH₂(CF₂)₂F, CH₂═CHCOO—CH₂CH₂CH₂ (CF)₂H, CH₂═CHCOO—CH₂(CF₂)₄H, CH₂═CHCOO—CH₂CH₂(CF₂)₃H, CH₂═CHCOO—CH₂CH₂CF(CF₃)—[OCF₂—CF(CF₃)]_{z}—OC₃F₇, CH₂═CHCOO—CH₂CH₂CF₂CF₂(CF₃)—[OCF₂—CF(CF₃)]₂—OC₃F₇.

Among these, the vinyl polymers represented by the formula given below are preferred:

CH₂═CHCOO—CH₂CH₂(CF₂)₆F, CH₂═CHCOO—CH₂CH₂(CF₂)₈F, CH₂═CCH₃COO—CH₂CH₂(CF₂)₆F, CH₂═CCH₃COO—CH₂CH₂(CF₂)₈F, CH₂═CHCOO—CH₂CF₃, CH₂═CCH₃COO—CH₂CF₃.

The vinyl polymers represented by the formulae given below are particularly preferred:

CH₂═CHCOO—CH₂CF₃, CH₂═CCHCOO—CH₂CF₃.

The vinyl monomers (B) not containing any organofluorine groups in the molecule may be any monomers containing radical-polymerizable vinyl groups which are exemplified, for example, by methyl acrylate, methyl methacrylate, ethyl acrylate, ethyl methacrylate, n-propyl acrylate, n-propyl methacrylate, isopropyl acrylate, isopropyl methacrylate, and other lower alkyl acrylates or methacrylates; glycidyl acrylate, glycidyl methacrylate; n-butyl acrylate, n-butyl methacrylate, isobutyl acrylate, isobutyl methacrylate, tert-butyl acrylate, tert-butyl methacrylate, n-hexyl acrylate, n-hexyl methacrylate, n-hexyl acrylate, n-hexyl methacrylate, cyclohexyl acrylate, cyclohexyl methacrylate, 2-ethylhexyl acrylate, 2-ethylhexyl methacrylate, octyl acrylate, octyl methacrylate, lauryl acrylate, lauryl methacrylate, stearyl acrylate, stearyl methacrylate, and other higher acrylates and methacrylates; vinyl acetate, vinyl propionate, and other lower fatty acid vinyl esters; vinyl butyrate, vinyl caproate, vinyl 2-ethylhexanoate, vinyl laurate, vinyl stearate, and other higher fatty acid esters; styrene, vinyltoluene, benzyl acrylate, benzyl methacrylate, phenoxyethyl acrylate, phenoxyethyl methacrylate, vinylpyrrolidone, and other vinylaromatic monomers; dimethylaminoethyl acrylate, dimethylaminoethyl methacrylate, diethylaminoethyl acrylate, diethylaminoethyl methacrylate, and other aminovinyl monomers, acrylamide, methacrylamide, N-methylolacrylamide, N-methylolmethacrylamide, N-methoxymethylacrylamide, N-methoxy-methylmethacrylamide, isobutoxymethoxyacrylamide, isobutoxymethoxy-methacrylamide, N,N-dimethylacrylamide, N,N-dimethylmethacrylamide, and other vinylamide monomers; hydroxyethyl acrylate, hydroxyethyl methacrylate, acrylic acid hydroxypropyl alcohol, methacrylic acid hydroxypropyl alcohol, and other hydroxy vinyl monomers; acrylic acid, methacrylic acid, itaconic acid, crotonic acid, fumaric acid, maleic acid, and other vinylcarboxylic acid monomers; tetrahydrofurfuryl acrylic, tetrahydrofurfuryl methacrylate, butoxyethyl acrylate, butoxyethyl methacrylate, ethoxydiethylene glycol acrylate, ethoxydiethylene glycol methacrylate, polyethylene glycol acrylate, polyethylene glycol methacrylate, polypropylene glycol monoacrylate, polypropylene glycol monomethacrylate, hydroxybutyl vinyl ether, cetyl vinyl ether, 2-ethylhexyl vinyl ether and other vinyl monomers containing an ether bond; acryloxypropyltrimethoxysilane, methacryloxypropyltrimethoxysilane, polydimethylsiloxanes containing acryl or methacryl groups at one of the ends, polydimethylsiloxanes containing alkenylaryl groups at one of the ends and other silicone compounds containing unsaturated groups; butadiene; vinyl chloride; vinylidene chloride, acrylonitrile, methacrylonitrile; dibutyl fumarate; maleic anhydride; dodecylsuccinic anhydride; acryl glycidyl ether, methacryl glycidyl ether, 3,4-epoxycyclohexylmethyl acrylate, 3,4-epoxy-cyclohexylmethyl methacrylate, alkali metal salts, ammonium salts and organic amine salts of acrylic acid, of methacrylic acid, of itaconic acid, of crotonic acid, of fumaric acid, of maleic acid and of other radical-polymerizable unsaturated carboxylic acids, radical-polymerizable unsaturated monomers containing sulfonic acid groups, such as styrene sulfonic acid and also the alkali metal salts thereof, the ammonium salts thereof and the organic amine salts thereof; the quaternary ammonium salts derived from acrylic acid or from methacrylic acid, such as 2-hydroxy-3-methacryloxypropyltrimethylammonium chloride, methacrylic acid esters of a tertiary amine alcohol, such as the diethylamine ester of methacrylic acid and quaternary ammonium salts thereof.

In addition, it is also possible to use, by way of vinyl monomers (B), the polyfunctional vinyl monomers which are exemplified, for example, by trimethylolpropane triacrylate, trimethylolpropane trimethacrylate, pentaerythritol triacrylate, pentaerythritol trimethacrylate, ethylene glycol diacrylate, ethylene glycol dimethacrylate, tetraethylene glycol diacrylate, tetraethylene glycol dimethacrylate, polyethylene glycol diacrylate, polyethylene glycol dimethacrylate, 1,4-butanediol diacrylate, 1,4-butanediol dimethacrylate, 1,6-hexanediol diacrylate, 1,6-hexanediol dimethacrylate, neopentyl glycol diacrylate, neopentyl glycol dimethacrylate, trimethylolpropanetrioxyethyl acrylate, trimethylolpropanetrioxyethyl methacrylate, tris(2-hydroxyethyl)isocyanurate diacrylate, tris(2-hydroxyethyl)isocyanurate dimethacrylate, tris(2-hydroxyethyl)isocyanurate triacrylate, tris(2-hydroxyethyl)isocyanurate trimethacrylate, polydimethylsiloxane in which the two ends of the molecular chain are blocked with alkenylaryl groups, and other silicone compounds containing unsaturated groups.

As regards the ratio mentioned above in which the component (A) and the component (B) are copolymerized, the weight ratio of the compound (A) to the compound (B) should be within the range from 0.1:99.9 to 100:0, and preferably within the range 1:99 to 100:0. The carbosiloxane dendrimer (C) is represented by general formula (III) indicated above.

In the component (C), Y may be a radical-polymerizable organic group, the type of which is not subject to any special limitations provided that it is an organic group capable of undergoing a radical addition reaction.

Y may be selected, for example, from organic groups containing acrylic or methacrylic groups, organic groups containing an alkenylaryl group, or alkenyl groups having from 2 to 10 carbon atoms.

The organic groups containing acrylic or methacrylic groups may be represented by the general formula:

[chemical structure]

The alkenylaryl group may be represented by the formula:

[chemical structure]

In the formula above, R⁴ and R⁶ are hydrogen atoms or methyl groups, R⁵ and R⁸ are alkylene groups having from 1 to 10 carbon atoms, and R⁷ is an alkyl group having from 1 to 10 carbon atoms. The index “b” is an integer from 0 to 4, and “c” is 0 or 1.

Acryloxymethyl, 3-acryloxypropyl, methacryloxymethyl, 3-methacryloxypropyl, 4-vinylphenyl, 3-vinylphenyl, 4-(2-propenyl)phenyl, 3-(2-propenyl)phenyl, 2-(4-vinylphenyl)ethyl, 2-(3-vinylphenyl)enyl, vinyl, allyl, methallyl and 5-hexenyl are suggested by way of examples of such radical-polymerizable organic groups.

The “i” in formula (II), which is an integer from 1 to 10, is the number of generations of said silylalkyl group, in other words the number of times that the silylalkyl group is repeated.

Thus, the carbosiloxane dendrimer of this component with a generation number of 1 is represented by the general formula:

[chemical structure]

in which Y, R¹, R² and R³ are as above and R¹² is a hydrogen atom or as R¹ described above. The index “a¹” is an integer from 0 to 3, the average total of “a¹” per molecule being from 0 to 7.

The carbosiloxane dendrimers of this component with a generation number of 2 are represented by the general formula:

[chemical structure]

in which Y, R¹, R², R³ and R¹² are as above and the indices “a¹” and “a²” are integers from 0 to 3, the average total of “a¹” and “a²” per molecule being from 0 to 25.

The carbosiloxane dendrimers of this component with a generation number of 3 are represented by the general formula:

[chemical structure]

in which Y, R¹, R², R³ and R¹² are as above and the indices “a¹”, “a²” and “a³” are integers from 0 to 3, the average total of “a¹”, of “a²” and of “a³” per molecule being from 0 to 79.

The component (C) can be exemplified by carbosiloxane dendrimers represented by formulae of mean composition, represented below:

[chemical structure]

[chemical structure]

In particular, the carbosiloxane-dendrimer-derived unit comprises at least one tri[tri(trimethylsiloxy)silylethyldimethylsiloxy]silylpropyl carbosiloxane-dendrimer-derived unit corresponding to one of the formulae:

[chemical structure]

The carbosiloxane dendrimers of the component (C) may be prepared using the process for preparing the siloxane/silylalkylene branched copolymers described in document EP 1 055 674.

For example, they may be prepared by subjecting organic alkenyl silicone compounds and silicone compounds comprising hydrogen atoms linked to the silicon, represented by the general formula:

[chemical structure]

in which R¹ and Y are as defined above, to a hydrosilylation reaction.

For example, 3-methacryloxypropyltris(dimethylsiloxy)silane, 3-acryloxypropyltris(dimethylsiloxy)silane and 4-vinylphenyltris(dimethylsiloxy)silane are used as silicon compounds represented by the above formula. Vinyltris(trimethylsiloxy)silane, vinyltris(dimethylphenylsiloxy)silane and 5-hexenyltris(trimethylsiloxy)-silane are used as organosilicon alkenyl compounds. In addition, it is preferable to perform the hydrosilylation reaction in the presence of a transition metal catalyst such as chloroplatinic acid and the platinum/vinylsiloxane complex.

The copolymerization ratio of the component (C), in terms of its weight ratio relative to the total weight of compound (A) and of compound (B), should be within the range from 0.1:99.9 to 99.9:0.1, preferably within the range 1:99 to 99:1, and even more preferably within the range from 5:95 to 95:5.

Amino groups may be introduced into the side chains of the vinyl polymer using, included in the component (B), vinyl monomers containing amino groups, such as dimethylaminoethyl acrylate, dimethylaminoethyl methacrylate, diethylaminoethyl acrylate and diethylaminoethyl methacrylate, followed by performing a modification with potassium acetate monochloride, ammonium acetate monochloride, the aminomethylpropanol salt of monochloroacetic acid, the triethanolamine salt of monobromoacetic acid, sodium monochloropropionate, and other alkali metal salts of halogenated fatty acids; otherwise, carboxylic acid groups may be introduced into the side chains of the vinyl polymer using, included in the component (B), vinyl monomers containing carboxylic acids, such as acrylic acid, methacrylic acid, itaconic acid, crotonic acid, fumaric acid and maleic acid, and the like, followed by neutralizing the product with triethylamine, diethylamine, triethanolamine and other amines.

A fluorinated vinyl polymer may be one of the polymers described in the examples of application WO 03/045337.

According to one preferred embodiment, a grafted vinyl polymer for the purpose of the invention may be carried in an oil or a mixture of oil(s), preferably volatile oil(s), in particular selected from silicone oils and hydrocarbon-based oils and mixtures thereof. According to one particular embodiment, a silicone oil suitable for the invention may be cyclopentasiloxane.

According to another particular embodiment, a hydrocarbon-based oil suitable for the invention may be isododecane.

According to a preferred embodiment, the vinyl polymer grafted with at least one carboxiloxane-dendrimer-derived unit is acrylates/polytrimethylsiloxymethacrylate copolymer.

The vinyl polymers grafted with at least one carbosiloxane-dendrimer-derived unit that may be particularly suitable for the present invention are the polymers sold under the names TIB 4-100, TIB 4-101, TIB 4-120, TIB 4-130, TIB 4-200, FA 4003, FA 4002 ID (TIB 4-202), TIB 4-220 and FA 4001 CM (TIB 4-230) by the company Dow Corning.

A composition of the invention may comprise a film forming polymer of vinyl polymer grafted with a carbosiloxane dentrimer in a content with respect to active material ranging from 0.05% to 20% by weight, preferably ranging from 0.1% to 15% by weight, and preferentially ranging from 0.5% to 5% by weight, relative to the total weight of the composition.

Pigment(s)

The composition of the present invention comprises at least one pigment.

The term “pigments” should be understood as meaning white or coloured, inorganic (mineral) or organic particles, which are insoluble in the liquid organic phase, and which are intended to colour and/or opacify the composition and/or the deposit produced with the composition.

The pigments may be chosen from mineral pigments, organic pigments and composite pigments (i.e. pigments based on mineral and/or organic materials).

The pigments may be chosen from monochromatic pigments, lakes and pigments with an optical effect, for instance goniochromatic pigments and nacres.

The mineral pigments may be chosen from metal oxide pigments, chromium oxides, iron oxides (black, yellow, red), titanium dioxide, zinc oxides, cerium oxides, zirconium oxides, chromium hydrate, manganese violet, Prussian blue, ultramarine blue, ferric blue, metal powders such as aluminium powders and copper powder, and mixtures thereof.

Organic lakes are organic pigments formed from a dye attached to a substrate.

The lakes, which are also known as organic pigments, may be chosen from the materials below, and mixtures thereof:
cochineal carmine;
organic pigments of azo dyes, anthraquinone dyes, indigoid dyes, xanthene dyes, pyrene dyes, quinoline dyes, triphenylmethane dyes or fluorane dyes.

Among the organic pigments that may in particular be mentioned are those known under the following names: D&C Blue No. 4, D&C Brown No. 1, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5, FD&C Yellow No. 6;
the organic lakes may be insoluble sodium, potassium, calcium, barium, aluminium, zirconium, strontium or titanium salts of acidic dyes such as azo, anthraquinone, indigoid, xanthene, pyrene, quinoline, triphenylmethane or fluorane dyes, these dyes possibly comprising at least one carboxylic or sulfonic acid group.

The organic lakes may also be supported on an organic support such as rosin or aluminium benzoate, for example.

Among the organic lakes, mention may be made in particular of those known under the following names: D&C Red No. 2 Aluminium lake, D&C Red No. 3 Aluminium lake, D&C Red No. 4 Aluminium lake, D&C Red No. 6 Aluminium lake, D&C Red No. 6 Barium lake, D&C Red No. 6 Barium/Strontium lake, D&C Red No. 6 Strontium lake, D&C Red No. 6 Potassium lake, D&C Red No. 7 Aluminium lake, D&C Red No. 7 Barium lake, D&C Red No. 7 Calcium lake, D&C Red No. 7 Calcium/Strontium lake, D&C Red No. 7 Zirconium lake, D&C Red No. 8 Sodium lake, D&C Red No. 9 Aluminium lake, D&C Red No. 9 Barium lake, D&C Red No. 9 Barium/Strontium lake, D&C Red No. 9 Zirconium lake, D&C Red No. 10 Sodium lake, D&C Red No. 19 Aluminium lake, D&C Red No. 19 Barium lake, D&C Red No. 19 Zirconium lake, D&C Red No. 21 Aluminium lake, D&C Red No. 21 Zirconium lake, D&C Red No. 22 Aluminium lake, D&C Red No. 27 Aluminium lake, D&C Red No. 27 Aluminium/Titanium/Zirconium lake, D&C Red No. 27 Barium lake, D&C Red No. 27 Calcium lake, D&C Red No. 27 Zirconium lake, D&C Red No. 28 Aluminium lake, D&C Red No. 30 lake, D&C Red No. 31 Calcium lake, D&C Red No. 33 Aluminium lake, D&C Red No. 34 Calcium lake, D&C Red No. 36 lake, D&C Red No. 40 Aluminium lake, D&C Blue No. 1 Aluminium lake, D&C Green No. 3 Aluminium lake, D&C Orange No. 4 Aluminium lake, D&C Orange No. 5 Aluminium lake, D&C Orange No. 5 Zirconium lake, D&C Orange No. 10 Aluminium lake, D&C Orange No. 17 Barium lake, D&C Yellow No. 5 Aluminium lake, D&C Yellow No. 5 Zirconium lake, D&C Yellow No. 6 Aluminium lake, D&C Yellow No. 7 Zirconium lake, D&C Yellow No. 10 Aluminium lake, FD&C Blue No. 1 Aluminium lake, FD&C Red No. 4 Aluminium lake, FD&C Red No. 40 Aluminium lake, FD&C Yellow No. 5 Aluminium lake and FD&C Yellow No. 6 Aluminium lake.

Mention may also be made of liposoluble dyes, such as, for example, Sudan Red, DC Red 17, DC Green 6, β-carotene, soybean oil, Sudan Brown, DC Yellow 11, DC Violet 2, DC Orange 5 and quinoline yellow.

The chemical substances corresponding to each of the organic colorants cited above are mentioned in the publication “International Cosmetic Ingredient Dictionary and Handbook”, 1997 edition, pages 371 to 386 and 524 to 528, published by “The Cosmetic, Toiletries and Fragrance Association”, the content of which is incorporated into the present patent application by way of reference.

The pigments may also have been subjected to a hydrophobic treatment.

The hydrophobic treatment agent may be chosen from silicones such as methicones, dimethicones, alkoxysilanes and perfluoroalkylsilanes; fatty acids such as stearic acid; metal soaps such as aluminium dimyristate, the aluminium salt of hydrogenated tallow glutamate, perfluoroalkyl phosphates, perfluoroalkylsilanes, perfluoroalkylsilazanes, polyhexafluoropropylene oxides, polyorganosiloxanes comprising perfluoroalkyl perfluoropolyether groups, and amino acids; N-acylamino acids or salts thereof; lecithin, isopropyl triisostearyl titanate, and mixtures thereof.

The N-acylamino acids can comprise an acyl group containing from 8 to 22 carbon atoms, such as, for example, a 2-ethylhexanoyl, caproyl, lauroyl, myristoyl, palmitoyl, stearoyl or cocoyl group. The salts of these compounds may be aluminium, magnesium, calcium, zirconium, zinc, sodium or potassium salts. The amino acid may be, for example, lysine, glutamic acid or alanine.

The term “alkyl” mentioned in the compounds cited above in particular denotes an alkyl group containing from 1 to 30 carbon atoms and preferably containing from 5 to 16 carbon atoms.

Hydrophobically treated pigments are described in particular in patent application EP-A-1 086 683.

Preferably, the pigment used in the present invention is hydrophobic treated pigments.

More preferably, the pigment used in the present invention is selected from pigments treated with silica and alumina, perfluorooctyl triethoxysilane and aluminium hydroxide, alumina and isopropyl titanium triisostearate, aluminium hydroxide and hydrogenated lecithin, disodium stearoyl glutamate and aluminium hydroxide, or a mixture thereof.

Advantageously, the pigment is present in an amount ranging from 1% to 30% by weight, preferably from 2% to 25% by weight, and more preferably from 5% to 20% by weight, relative to the total weight of the composition.

Aqueous Phase

The composition according to the present invention comprise at least one aqueous phase.

Preferably the aqueous phase is a continuous aqueous phase.

The at least one aqueous phase comprises water.

The aqueous phase may also comprise water-miscible organic solvents (at room temperature: 25° C.), for instance monoalcohols containing from 2 to 6 carbon atoms, such as ethanol or isopropanol; polyols especially containing from 2 to 20 carbon atoms, preferably containing from 2 to 10 carbon atoms and preferentially containing from 2 to 6 carbon atoms, such as glycerol, propylene glycol, butylene glycol, pentylene glycol, hexylene glycol, dipropylene glycol or diethylene glycol; glycol ethers (especially containing from 3 to 16 carbon atoms) such as mono-, di- or tripropylene glycol (C₁-C₄)alkyl ethers, mono-, di- or triethylene glycol (C₁-C₄)alkyl ethers, and mixtures thereof.

The aqueous phase may also comprise any water-soluble or water-dispersible compound that is compatible with an aqueous phase, such as gelling agents, thickeners or surfactants, and mixtures thereof.

In particular, a composition of the present invention may comprise the aqueous phase in a content ranging from 20%% to 95%% by weight, especially from 30% to 85% by weight relative to the total weight of the composition.

According to a preferred embodiment, water is present in an amount ranging from 10% to 80% by weight, preferably 20% to 70% by weight, relative to the total weight of the composition.

According to a preferred embodiment, the present invention relates to a composition for making up keratin materials, in particular the skin, comprising, in a continuous aqueous phase, relative to the total weight of the composition:
(i) from 0.3% to 2.0% by weight of acrylates/beheneth-25 methacrylate copolymer;
(ii) from 0.5% to 5% by weight of acrylates/polytrimethylsiloxymethacrylate copolymer; and
(iii) from 5% to 20% by weight of at least one pigment.

Oil(s)

A composition of the present invention may comprise at least one oil.

The term “oil” refers to any fatty body in liquid form at room temperature (20-25° C.) and atmospheric pressure.

These oils may be of animal, plant, mineral or synthetic origin.

The oils may be volatile or non-volatile.

The term “volatile oil” refers to any non-aqueous medium capable of evaporating from the skin or lips, in less than one hour, at room temperature (20-25° C.) and atmospheric pressure (760 mmHg). The volatile oil is a volatile cosmetic oil, liquid at room temperature. More specifically, a volatile oil has an evaporation rate of from 0.01 to 200 mg/cm²/min.

The term “non-volatile oil” is intended to mean an oil remaining on the skin or keratin fiber at ambient temperature and atmospheric pressure. More specifically, a non-volatile oil has an evaporation rate strictly below 0.01 mg/cm²/min.

To measure this evaporation rate, 15 g of oil or a mixture of oils to be tested are introduced into a crystallizer, 7 cm in diameter, placed on a scale located in a large 0.3 m³ chamber temperature-controlled at a temperature of 25° C., and humidity-controlled with a relative humidity of 50%. The liquid is left to evaporate freely, without stirring, by providing ventilation with a fan (PAPST-MOTOREN, reference 8550 N, rotating at 2700 rpm) positioned vertically above the crystallizer containing the solvent, with the blades directed toward the crystallizer and at a distance of 20 cm from the base of the crystallizer. The mass of oil remaining in the crystallizer is measured at regular intervals. The evaporation rates are expressed in mg of oil evaporated per surface area unit (cm²) and per time unit (minute).

The oils that are suitable for the present invention may be additional hydrocarbon-based oils different from the ingredients mentioned above, silicone-based or fluorine-based.

According to the present invention, the term “silicone oil” refers to an oil including at least one silicon atom, and in particular at least on Si—O group.

The term “fluorine oil” refers to an oil including at least one fluorine atom.

The term “hydrocarbon oil” refers to an oil containing primarily hydrogen and carbon atoms.

The oils may optionally include oxygen, nitrogen, sulfur and/or phosphorus atoms, for example, in the form of hydroxyl or acid radicals.

As additional hydrocarbon oils, mentions may be made of volatile oils such as C₈-C₁₆ alkanes, for example dodecane, tetradecane; non-volatile oils such as hydrocarbon oils of animal origin, hydrocarbon oils of plant origin, linear or branched hydrocarbons of mineral or synthetic origin, synthetic ethers having from 10 to 40 carbon atoms; polyol esters and pentaerythritol esters, esters of diol dimers and diacid dimers, copolymers of a diol dimer and of a diacid dimer and esters thereof, copolymers of polyols and of diacid dimers, fatty alcohols that are liquid at ambient temperature, C₁₂-C₂₂, higher fatty acids, oils of higher molar mass having in particular a molar mass ranging from approximately 400 to approximately 10,000 g/mol, lipophilic polymers, hydroxylated esters, aromatic esters, esters of C₂₄-C₂₈ branched fatty acids or fatty alcohols, and mixtures thereof.

As silicone oils, mention may be made of:

linear or cyclic volatiles oils, especially those with a viscosity of less than or equal to 8 centistokes (cSt) (8×10⁻⁶ m²/s), and especially containing from 2 to 10 silicon atoms and in particular from 2 to 7 silicon atoms, these silicones optionally comprising alkyl or alkoxy groups containing from 1 to 10 carbon atoms;

linear or cyclic non-volatile polydimethylsiloxanes (PDMS); polydimethylsiloxanes comprising alkyl, alkoxy or phenyl groups, which are pendant or at the end of a silicone chain, these groups containing from 2 to 24 carbon atoms; for example cyclohexasiloxane, which is commercially available under the tradename Silsoft 1217 sold by the company Momentive Performance Materials.

phenyl silicones, for instance phenyl trimethicones, phenyl dimethicones, phenyl trimethylsiloxy diphenyl siloxanes, diphenyl dimethicones, diphenyl methyldiphenyl trisiloxanes and 2-phenylethyl trimethylsiloxy silicates, for example the product phenyl trimethicone sold by the company Dow Corning under the tradename Dow Corning 556 Cosmetic Grade Fluid.

and mixtures thereof.

Preferably, the oil is selected from linear or cyclic non-volatile polydimethylsiloxanes, phenyl silicones, more preferably selected from cyclohexasiloxane, phenyl trimethicone, or a mixture thereof.

According to a preferred embodiment, when exists, the oil is present in the composition of the present invention in an amount ranging from 0.01% to 20% by weight, preferably from 0.1% to 15% by weight, relative to the total weight of the composition.

Adjuvants

In a known way, all the compositions of the present invention can comprise one or more of the adjuvants usual in the cosmetic and dermatological fields: hydrophilic or lipophilic gelling and/or thickening agents; fillers; moisturising agents; emollients; hydrophilic or lipophilic active principles; agents for combating free radicals; sequestering agents; UV-screening agents; antioxidants; preservatives; basifying or acidifying agents; fragrances; plant extracts; and their mixtures. The amounts of these various adjuvants are those conventionally used in foundations.

Of course, a person skilled in the art will take care to choose the optional adjuvants added to the composition according to the present invention so that the advantageous properties intrinsically attached to the composition according to the present invention are not, or not substantially, detrimentally affected by the envisaged addition.

Galenic Form

According to one embodiment, the composition according to the present invention is in the form of a liquid composition. Nevertheless, it is to be understood that a person skilled in the art can choose the appropriate presentation form, on the basis of his/her general knowledge, taking into account the nature of the constituents used, for example, their solubility in the vehicle, and the application envisaged for the composition.

According to a preferred embodiment, the composition according to the present is in form of oil dispersion in a continuous aqueous phase.

Method and Use

The composition of the present invention can be generally prepared according to the general knowledge of a person skilled in the art. Nevertheless, it is to be understood that a person skilled in the art can choose its method of preparation, on the basis of his/her general knowledge, taking into account the nature of the constituents used, for example, their solubility in the vehicle, and the application envisaged for the composition.

The composition of the present invention can be used for a non-therapeutic process, such as a cosmetic process or method, for making up/caring for a keratin material, such as the skin, by being applied to the skin.

The method according to the present invention can improve skin sensory, in particularly water breaking feeling, without deteriorating other beneficial effects like easy application to the e.g. skin.

### Examples

The examples that follow are aimed at illustrating the compositions and processes according to the present invention, but are not in any way a limitation of the scope of the present invention.

I. Preparation

The present invention formulas 1, 2, and comparative formulas 1 were prepared.

| | | % by weight of active | |
|---|---|---|---|
| | | matters | |
| | | Invention | Comparative |
| Phase | INCI name | formula 1 | formula 1 |
| A | WATER | QS to 100 | QS to 100 |
| A | PEG-11 METHYL ETHER | 0.5 | 0.5 |
| | DIMETHICONE (and) | | |
| | PPG-5-BUTETH-5 (KF 351 A from | | |
| | Shin Etsu) | | |
| A | PHENOXYETHANOL | 0.7 | 0.7 |
| A | CHLORPHENESIN | 0.2 | 0.2 |
| A | CAPRYLYL GLYCOL (Dermosoft | 0.3 | 0.3 |
| | Octiol from Dr Straetmans) | | |
| A | OCTYLDODECYL MYRISTATE | 2 | 2 |
| | (Myristate DE 2 Octyl-dodecyle from | | |
| | Gattefosse) | | |
| A | GLYCERYL STEARATE (and) | 1 | 1 |
| | PEG-100 STEARATE (SP Arilancel | | |
| | from Croda) | | |
| B | ACRYLATES/BEHENETH-25 | 0.4 | 0.4 |
| | METHACRYLATE COPOLYMER | | |
| | (Volarest ™ FL-LQ-(RB) from Croda) | | |
| B | SODIUM HYDROXIDE | 0.06 | 0.06 |
| C | DIMETHICONE (and) ACRYLATES/ | 2.0 | 0 |
| | POLYTRIMETHYLSILOXYMETHA- | | |
| | CRYLATE COPOLYMER (and) | | |
| | DIMETHICONE (Dow Corning ® | | |
| | FA-4003 from Dow) | | |
| C | POLYURETHANE-35 | 0 | 2.0 |
| | (BAYCUSAN ® C 1004 from | | |
| | Covestro (BAYER)) | | |
| D | TITANIUM DIOXIDE (and) | 10 | 10 |
| | DISODIUM STEAROYL | | |
| | GLUTAMATE (and) ALUMINUM | | |
| | HYDROXIDE (NAI-WHITEA from | | |
| | MIYOSHI KASEI) | | |
| E | DIMETHICONE (Belsil DM 5 Plus | 5 | 5 |
| | from Wacker) | | |

The comparative formula 1 contains a film forming polymer Polyurethane-35, which is out of the scope of the present invention.

| | | Invention formula 2 |
|---|---|---|
| | | (% by weight of |
| Phase | INCI name | active matters) |
| A | WATER | QS to 100 |
| A | PEG-11 METHYL ETHER DIMETHICONE | 0.5 |
| | (and) PPG-5-BUTETH-5 (KF 351 A from Shin | |
| | Etsu) | |
| A | PHENOXYETHANOL | 0.7 |
| A | CHLORPHENESIN | 0.2 |
| A | CAPRYLYL GLYCOL (Dermosoft Octiol from | 0.3 |
| | Dr Straetmans) | |
| A | OCTYLDODECYL MYRISTATE (Myristate | 2 |
| | DE 2 Octyl-dodecyle from Gattefosse) | |
| A | GLYCERYL STEARATE (and) PEG-100 | 1 |
| | STEARATE (SP Arilancel from Croda) | |
| B | ACRYLATES/BEHENETH-25 | 0.4 |
| | METHACRYLATE COPOLYMER (Volarest ™ | |
| | FL-LQ-(RB) from Croda) | |
| B | SODIUM HYDROXIDE | 0.06 |
| C | DIMETHICONE (and) | 1 |
| | ACRYLATES/POLYTRIMETHYLSILOXYME | |
| | THACRYLATE COPOLYMER (and) | |
| | DIMETHICONE (Dow Corning ® FA-4003 | |
| | from Dow) | |
| D | TITANIUM DIOXIDE (and) DISODIUM | 10 |
| | STEAROYL GLUTAMATE (and) ALUMINUM | |
| | HYDROXIDE (NAI-WHITEA from MIYOSHI | |
| | KASEI) | |
| D | DIMETHICONE (Belsil DM 5 Plus from | 5 |
| | Wacker) | |
| E | BORON NITRIDE (Boron Nitride SHP 3 from | 1 |
| | Mizushima Ferroalloy) | |

The present invention and comparative formulas were prepared following the preparation methods which are conventionally known to the man skilled in the art.

II. Evaluation of the Present Invention and Comparative Formulas

The present invention and comparative formulas listed above were evaluated using the following protocols.

Method for Evaluating the Stability Over Time

The present invention and comparative formulas were kept under 4° C., 37° C., and 45° C. for 2 months.

Method for Evaluating the Hydration Effect

The evaluation was performed as follows.

Step 1: Pick up same quantity (around 0.1 g) of the invention and comparative formulas, respectively, then using the pad of the middle finger and apply the product on inner forearm.

Step 2: Spread the invention and comparative formulas, respectively, simultaneously in order to have a good distribution of the product over same area.

Step 3: Observe the hydration effect. If it has been observed a significant difference between an area of the skin treated with a formula compared with an untreated area, we can say that the treated skin was better moisturized than the untreated skin.

Method for Evaluating the Cosmetic Properties

The coverage and long-lasting effect of the invention and comparative formulas were evaluated.

To measure the coverage of the formulas listed above, a wet film of 50 μm thickness of each of the formulas was made on standard black and white contrast card. Film was then dried for 24 hours at room temperature. Chromameter CR-400 was used to measure the L* value on both white side and black side.

Coverage=(L*black+16)\hat{ }3/(L*white+16)\hat{ }3*100%

L*black means L* value measured on black card.

L*white means L* value measured on white card.

To observe the long lasting property, the film integrity when in contact with artificial sebum was measured:

place 50 mg of each of the invention and comparative formulas on a glass slide, respectively, add one drop of artificial sebum on top of the formulas in the glass slide, observe the film integrity immediately and after 60 min.

The artificial sebum is made up of 20% of oleic acid, 1% of oleth-10, 0.2% of methylparaben and 78.8% of water.

The hydration effect and film integrity effect were scored by 5 of panelists.

Score 5: at least 80% of scientists/consumers consider the effect being good and expected;

Score 4: from 60% to 80% (not included) of scientists/consumers consider the effect being good and expected; Score 3: at least 50% of scientists/consumers consider the effect being good and expected;

Score 2: more than 60% of scientists/consumers consider the effect being poor and unacceptable;

Score 1: more than 80% of scientists/consumers consider the effect being poor and unacceptable

The results of the evaluation are as follow.

| | Invention formula | | Comparative |
|---|---|---|---|
| Item | 1 | 2 | formula 1 |
| Stability | stable | stable | stable |
| Film integrity | 5 | 4 | 1, film destroyed, |
| | | | pigment cluster |
| | | | occurred |
| Coverage | 76% | 74% | 74% |
| Hydration | 4 | 5 | 2 |
| property | | | |

From the results above, it is observed that compared to comparative formula 1 with film former Polyurethane-35, invention formulas 1 and 2 both exhibit good stability as well as coverage. Besides, they both have significant better film integrity and hydration properties.

Therefore, it is obvious from the results listed above, that the invention formulas have improved cosmetic properties and hydration sensory, comparing to the comparative formula.

1. A composition for caring for and/or making up keratin materials, comprising, in a continuous aqueous phase,
(i) at least one associative copolymer of (meth)acrylic acid, and of (C₁-C₆)alkyl ester of (meth)acrylic acid;
(ii) at least one film forming polymer chosen from vinyl polymer grafted with a carbosiloxane dentrimer; and
(iii) at least one pigment.

2. The composition according to claim 1, wherein the at least one associative copolymer (i) is obtained from copolymerization between 1) and 2):
1) at least one ethylenically unsaturated mono or dicarboxylic acid monomer substituted by at least one, linear or branched, (C₁-C₁₀)alkyl group; and
2) at least one associative monomer which is an ester of formula (I):
A-O-(Alk-O)_{z}—(CH₂)_{w}—R^{a} (I)
wherein:
A represents an ethylenically unsaturated acyclic residue, optionally containing an additional carboxylic group or it salt, wherein said additional carboxylic group may be esterified with a, linear or branched, (C₁-C₂₀)alkyl group;
R^{a} represents an alkyl, a linear or branched, (C₁-C₃₀)alkyl group, alkylaryl or arylalkyl group having from 1 to 30 carbon atoms wherein the alkyl group is linear or branched;
Alk represents a linear or branched (C₁-C₆)alkylene group;
z is an integer between 0 and 100,
w is an integer between 0 and 30; with the proviso that formula (I) contains at least one carboxyl group C(O)OH, or C(O)O-Q⁺ wherein Q⁺ represents cation selected from the group consisting of an alkali metal, alkaline earth metal and ammonium.

3. The composition according to claim 1, wherein the at least one associative copolymer (i) is obtained by polymerization between at least one ethylenically unsaturated mono or dicarboxylic acid monomer (1a) and at least one associative monomer which is an ester of formula (I) as defined by formula (2a): [chemical structure]
formulas (1a) and (2a) wherein:
R′ₐ and R′_{b}, identical or different, represent a hydrogen atom, or a linear or branched (C₁-C₆)alkyl group;
R′_{c} and R″_{c} identical or different, represent a hydrogen atom, a linear or branched (C₁-C₆)alkyl group, a C(O)OX group, or a -alk-C(O)OX group, wherein X represents a hydrogen atom, an alkali metal, an alkaline earth metal, or ammonium, and -alk- represents a (C₁-C₆)alkylene group;
R₁ represents a hydrogen atom, an alkali metal, an alkaline earth metal, or a (C₁-C₆)alkyl group;
R₂ represents a, linear or branched, (C₆-C₄₀)alkyl group;
n is an integer between 5 and 100;
being understood that (1a) or (2a) contain at least one carboxyl group C(O)OH, or C(O)O-Q⁺ wherein Q⁺ represents cation selected from the group consisting of an alkali metal, an alkaline earth metal, and ammonium.

4. The composition according to claim 1, wherein the at least one associative copolymer (i) contains units (Ia) and/or (I′a): [chemical structure]
wherein:
R′ₐ, R′_{b}, R′_{c}, and R″_{c} are as defined in claim 3;
x represents an integer between 100 and 10000;
y represents an integer between 100 and 10000;
and x+y represents an integer between 200 and 20000.

5. The composition according to claim 1, wherein the at least one associative copolymer (i) is selected from the group consisting of acrylates/beheneth-25 methacrylate copolymer, acrylates/steareth-20 methacrylate copolymer, acrylates/palmeth-25 acrylate copolymer, acrylates/steareth-20 itaconate copolymer, acrylates/ceteth-20 itaconate copolymer, acrylates/ceteth-20 methacrylate copolymer, acrylate/beheneth-25 itaconate copolymer, acrylate/palmeth-25 methacrylates copolymer, acrylate/steareth-50 acrylate copolymer, acrylates/palmeth-25 itaconate copolymer, and mixtures thereof.

6. The composition according to claim 1, wherein the associative copolymer (i) is present in an amount ranging from 0.05 to 2.5% by weight relative to the total weight of the composition.

7. The composition according to claim 1, in which the vinyl polymer (ii) having at least one carbosiloxane-dendrimer-derived unit has a side molecular chain containing a carbosiloxane dendrimer structure, and is derived from the polymerization:
(A) of 0 to 99.9 parts by weight of a vinyl monomer; and
(B) of 100 to 0.1 parts by weight of a carbosiloxane dendrimer containing a radical-polymerizable organic group, represented by the general formula: [chemical structure]
in which Y represents a radical-polymerizable organic group, R¹ represents an aryl group or an alkyl group having from 1 to 10 carbon atoms, and Xⁱ represents a silylalkyl group which, when i=1, is represented by the formula: [chemical structure]
in which R¹ is as defined above, R² represents an alkylene group having from 2 to 10 carbon atoms, R³ represents an alkyl group having from 1 to 10 carbon atoms, X represents a hydrogen atom, an alkyl group having from 1 to 10 carbon atoms, an aryl group, or the silylalkyl group as defined above with i=i+1; i is an integer from 1 to 10 which represents the generation of said silylalkyl, and a′ is an integer from 0 to 3; where said radical-polymerizable organic group contained in the component (B) is selected from:
organic groups containing a methacrylic group or an acrylic group and which are represented by the formula: [chemical structure]
in which R⁴ represents a hydrogen atom or an alkyl group, R⁵ represents an alkylene group having from 1 to 10 carbon atoms; and
organic groups containing a styryl group and which are represented by the formula: [chemical structure]
in which R⁶ represents a hydrogen atom or an alkyl group, R⁷ represents an alkyl group having from 1 to 10 carbon atoms, R⁸ represents an alkylene group having from 1 to 10 carbon atoms, b is an integer from 0 to 4, and c is 0 or 1, such that, if c is 0, —(R⁸)_{c}— represents a bond.

8. The composition according to claim 1, in which the vinyl polymer (ii) having at least one carbosiloxane-dendrimer-derived unit is derived from the copolymerization:
(A) of vinyl monomers containing organofluorine groups in the molecule,
(B) optionally of vinyl monomers not containing any organofluorine groups in the molecule, and
(C) of carbosiloxane dendrimers having radical-polymerizable organic groups represented by general formula (III): [chemical structure]
in which Y is a radical-polymerizable organic group, R¹ is an aryl or alkyl group having from 1 to 10 carbon atoms and Xⁱ is a silylalkyl group represented by formula (II) below: [chemical structure]
in which R¹ is as above, R² is an alkylene group having from 1 to 10 carbon atoms, R³ is an alkyl group having from 1 to 10 carbon atoms, and Xⁱ⁺¹ is a group selected from the group comprising hydrogen atoms, aryl groups and alkyl groups having up to 10 carbon atoms, and silylalkyl groups Xⁱ mentioned above where the “i” is an integer from 1 to 10 indicating the generation of said silylalkyl group beginning in each carbosiloxane dendritic structure with a value of 1 for the group Xⁱ in formula (III), and the index “aⁱ” is an integer from 0 to 3,
said vinyl polymer (ii) having a copolymerization ratio of the component (A) to the component (B) of 0.1 to 100:99.9 to 0% by weight, and a copolymerization ratio of the sum of the component (A) and of the component (B) to the component (C) of 0.1 to 99.9:99.9 to 0.1% by weight.

9. The composition as claimed in claim 8, in which the radical-polymerizable organic group Y in the component (C) is a group selected from the group comprising:
organic groups containing acrylic or methacrylic groups represented by the general formula: [chemical structure]
in which R⁴ is a hydrogen atom or methyl, and R⁵ is an alkylene group having from 1 to 10 carbon atoms
or [chemical structure]
in which R⁴ and R⁵ are as defined above,
organic groups containing alkenylaryl groups represented by the general formula: [chemical structure]
in which R⁶ is a hydrogen atom or methyl, R⁷ is an alkyl group having from 1 to 10 carbon atoms, R⁸ is an alkylene group having from 1 to 10 carbon atoms, “b” is an integer from 0 to 4 and “c” is 0 or 1,
and alkenyl groups having from 2 to 10 carbon atoms.

10. The composition as claimed in claim 1, in which the vinyl polymer (ii) having at least one carbosiloxane-dendrimer-derived unit comprises at least one tri[tri(trimethylsiloxy)silylethyldimethylsiloxy]silylpropyl carbosiloxane-dendrimer-derived unit, corresponding to one of the [chemical structure]
formulae:

[chemical structure]

11. The composition of claim 1, wherein the film forming polymer of vinyl polymer (ii) grafted with a carbosiloxane dentrimer in a content with respect to active material ranging from 0.05% to 20% by weight relative to the total weight of the composition.

12. The composition of claim 1, wherein the pigment is hydrophobic treated pigment, selected from surface coatings with silica and alumina, perfluorooctyl triethoxysilane and aluminium hydroxide, alumina and isopropyl titanium triisostearate, aluminium hydroxide and hydrogenated lecithin, disodium stearoyl glutamate and aluminium hydroxide.

13. The composition of claim 1, wherein the pigment is present in an amount ranging from 1% to 30% by weight relative to the total weight of the composition.

14. A composition for making up keratin materials, comprising, in a continuous aqueous phase, relative to the total weight of the composition:
(i) from 0.3% to 2.0% by weight of acrylates/beheneth-25 methacrylate copolymer;
(ii) from 0.5% to 5% by weight of acrylates/polytrimethylsiloxymethacrylate copolymer; and
(iii) from 5% to 20% by weight of at least one pigment.

15. A non-therapeutic cosmetic process for caring for and/or making up a keratin material, comprising the application, to the surface of the keratin material, of the composition according to claim 1.
